(19) United States
(12) Patent Application Publication
(10) Pub. No.: US 2026/0215913 A1
(43) Pub. Date: Jul. 30, 2026

(54) AORTIC VALVE PROSTHESIS

(71) Applicant: TriBio GmbH, Heilbronn (DE)

(72) Inventors: Ulrich SCHÄFER, Hamburg (DE); Hans-Hinrich SIEVERS, Kronshagen (DE); Georg MATHEIS, Heilbronn (DE)

(21) Appl. No.: 19/148,095

(22) PCT Filed: Jan. 11, 2024
(86) PCT No.: PCT/DE2024/035000

§ 371 Date: Jul. 15, 2025

(30) Foreign Application Priority Data
Jan. 16, 2023 (DE) 10 2023 200 296.5

(51) Int. Cl. A61F 2/24; A61F 2/95

(52) U.S. Cl. CPC A61F 2/2418; A61F 2/2427; A61F 2002/9505; A61F 2230/0093

(57) ABSTRACT

An aortic valve prosthesis is configured for a transcatheter implantation, with a valve frame (4) which can be folded together and having three commissures (6), between which three valve leaflets (2) are arranged. The commissures (6) at their ends each merge into a commissure holding bow (14), the ends of the commissure holding bows (14) which are away from the commissures (6) are connected to one another in a common connection point (16). The connection point (16) forms a free end of the aortic valve prosthesis, on which end a holding element (22) is arranged. The holding element is configured in a manner such that it can be gripped by a tool.

### CROSS REFERENCE TO RELATED APPLICATIONS

This application is a United States National Phase Application of International Application PCT/DE2024/035000, filed Jan. 11, 2024, and claims the benefit of priority under 35 U.S.C. § 119 of German Application 10 2023 200 296.5, filed Jan. 16, 2023, the entire contents of which are incorporated herein by reference.

### TECHNICAL FIELD

The present invention pertains to an aortic valve prosthesis which is configured for a transcatheter implantation.

### BACKGROUND

Such a biological aortic valve prosthesis is known for example from EP 3 579 788 B1. Aortic valve prostheses of this type comprise a valve frame which can be folded together and on which three valve leaflets are attached, said valve leaflets being manufactured of a biological or synthetic material. The valve frame is generally folded together or pressed together with the valve leaflets, in a manner such that it can be implanted in the heart with the help of a catheter. On transcatheter implantation, the correct positioning of the heart valve prosthesis in the inside of the existing aortic valve is of great importance.

### SUMMARY

It is an object of the invention to invention to provide an improved aortic valve prosthesis which is configured for transcatheter implantation, said aortic valve prosthesis being able to be positioned in the heart in a very precise manner and, as the case may be, removed again after a complete implantation.

This object is achieved by an aortic valve prosthesis with the features which are specified in the description as well as by an assembly tool with the features which are specified. Preferred embodiments result from the subsequent description as well as the attached figures.

The aortic valve prosthesis according to the invention is configured for the replacement of the aortic valve in the heart by way of transcatheter implantation and for this comprises a collapsible valve frame which is preferably manufactured of a shape-memory metal and is self-expanding. This valve frame defines three commissures, between which three valve leaflets, preferably of biological material, are arranged in the usual manner. The valve frame in a known manner can be sheathed by a textile material and the valve leaflets are connected, preferably sewn, to the valve frame, in particular to the commissures and/or to the surrounding textile material. The commissures of the valve frame or of the stent framework are preferably movable or elastically deformable, in order to assist or simplify the movement of the valve leaflets on opening and closing the heart valve and in order to reduce the mechanical loading of the valve leaflets.

According to the invention, the commissures are configured such that they do not end at a free end, but at these otherwise free ends each merge into a commissure holding bow. This means that the commissures merge in each case into a commissure holding bow at those ends which face the contact edges of the valve leaflets which define the opening of the valve. This is the end of the commissures which is downstream in the flow direction in the heart. The ends of the commissure holding bows which are away from the commissures, which is to say the ends which are downstream in the flow direction are connected to one another in a common connection point. The connection point defines a free end of the aortic valve prosthesis. This free end is free after the implantation and does not serve as an engagement point for fastening elements or fastening structures. This means that the connection point in particular in the implanted state comprises no further connection to the surrounding tissue. The aortic valve prosthesis according to the invention is herewith preferably fixed to the surrounding tissue only at the one upstream axial end which is away from the connection point. At this upstream region or end, the aortic valve prosthesis preferably has a stiffness in the radial direction which is greater in comparison to the downstream end. This fastening region which is situated upstream anchors itself to the surrounding tissue—the aortic annulus and other anatomical structures which are situated in the vicinity—preferably by way of self-expansion and on implantation can additionally be pressed radially outwards in an active manner in order to thus clamp itself on surrounding tissue. A holding element which is configured such that it can be gripped by a tool is arranged at the free end of the aortic valve prosthesis which is defined by the connection point. This permits the aortic valve prosthesis to be gripped again on implantation or after this, after it has already been released from the catheter, in order to be able to reposition it or correct the positioning. Thus the implantation can be significantly improved since the aortic valve prosthesis can be positioned in a more precise manner. In the extreme case, the aortic valve prosthesis can be pulled back again into the catheter and also be removed from the body again, for example in order to then subsequently introduce a better fitting aortic valve prosthesis.

In a possible embodiment, the aortic valve prosthesis can be configured with a supra-annular valve function, concerning which the valve leaflets come to lie above or downstream of the original aortic valve, e.g. 5 to 15 mm downstream of the original aortic valve.

The valve frame is preferably configured in a manner such that the firmness in the radial direction or the elastic expansion force in the radial direction is lower in the region of the downstream end of the valve frame, i.e. in particular in the region of the commissure holding bows, than in a region which is situated further upstream, in particular than in the fastening region. This simplifies the re-gripping and pulling of the aortic valve prosthesis into the catheter again, since the end which is to be gripped can be more easily deformed. Furthermore, this configuration favours the elastic deformability of the commissures and thus reduces the mechanical loading of the valve leaflets. An easier deformability by way of lower radial stiffness furthermore simplifies a possible post-treatment of the aortic valve prosthesis, e.g. with the help of a balloon or given a new valve implantation (TAVI in TAVI).

Preferably, the holding element is provided with at least one undercut and further preferably it is configured as a hook, ball or eyelet. A corresponding engagement element which is arranged on a matching tool can engage behind this holding element, in order to hence be able to pull the aortic valve prosthesis back into the catheter.

According to a further preferred embodiment of the invention, the valve frame comprises at least one valve protection structure each in the region between two commissures. This means that three valve protection structures are likewise provided on the aortic valve prosthesis, each in the region of one of the three valve leaflets. The valve protection structures are each configured in a manner such that they move radially inwards on folding together the valve frame. In this manner, the valve protection structures can prevent the middle regions of the valve leaflets, which is to say the regions of the valve leaflets which are situated between two commissures, from getting clamped between the commissures on folding together. Preferably, the valve leaflet is pressed radially inwardly or is held radially in the inside by way of the valve protection structure and is therefore protected from damage on folding together. In particular, this is advantageous if the aortic valve prosthesis after the unfolding in the heart is pulled back into the catheter again via the holding element and the connection bows, in order to once again position it anew. With regard to this folding-together again, there are no further tools available which fold together the valve leaflets in a defined manner. This is assumed by the valve protection structures and these ensure that the valve leaflets are not damaged in an undesired manner on folding together.

The valve frame is preferably configured such that the adjacent commissures at their end which is away from the holding bow merge into an arch which is to say merge into a frame arch which connects the commissures to one another. The valve leaflet is fastened in the region of this arch. Coming from a frame arch which connects two commissures, the valve protection structures preferably each extend between these commissures. This means that coming from the frame arch, the valve protection structure extends essentially in the same axial direction as the commissures, which is to say in the downstream direction. The end of the valve protection structure which is away from the frame arch or is distanced to this is then situated between the tips of two commissures, but further preferably has no direct connection to the commissures. This means that the valve protection structures each form a free element which is independent of the commissures and which particularly preferably can move independently of the commissures.

The valve protection structures are further preferably each formed by one or more webs or members of a lattice structure of the valve frame. Particularly preferably, they are formed as one piece with the lattice structure of the remaining valve frame. This permits the complete lattice structure of the valve frame including the valve protection structures to be manufactured in a single part manner as one component, for example to be cut from a pipe.

According to a further possible embodiment of the invention, the valve protection structures each at one end are connected to a structure holding bow which extends towards the connection point. The structure holding bows can each be led directly up to the connection point and be connected to this. Alternatively, it is possible for the structure holding bows to be connected to the commissure holding bows via connection members. Herein, the connection members run essentially in the circumferential direction. The structure holding bows herewith each connect a valve protection structure to the connection point in a direct or indirect manner. Herein, particularly preferably the structure holding bows are configured as one piece with the valve protection structure. The structure holding bows are further preferably shaped such that on pulling in the aortic valve prosthesis, they have the effect of the valve protection structures being moved radially inwards, as also of the commissures being moved radially inwards, by the commissure holding bows. Herein, the valve protection structures and their structure holding bows are further preferably configured such that they move radially inwards in front of the commissures. In this manner, one succeeds in the valve leaflets in their middle regions being moved radially inwards or being held radially inwards, and a defined folding-together of the valve leaflets is herewith ensured.

The commissure holding bows and/or the structure holding bows according to a further preferred embodiment each comprise at least one section which runs in an angled manner to the longitudinal axis of the aortic valve prosthesis at an angle of smaller than 90° and preferably smaller than 60°, said longitudinal axis running in the flow direction. Such an acute angle is preferably directed such that the sections which run in an angled manner approach one another towards the connection point. If the valve frame is gripped at the connection point and is pulled into a catheter, then the angled sections can thus slide of the face side of a catheter opening. In this manner, a radially inwardly directed force is generated by the oblique extension to the longitudinal direction, said force serving for radially inwardly deforming the valve frame and pressing it together, so that it is reduced again in its diameter with respect to the longitudinal axis and in the extreme case can be can be moved back into the catheter again.

In a further possible embodiment, the commissure holding bows and/or the structure holding bows can each comprise a first section which extends in the extension of the connected commissure or valve protection structure, which is to say essentially parallel to the longitudinal axis of the aortic valve prosthesis. According to this embodiment, the commissure holding bows and/or the structure holding bows furthermore comprise a second section which connects onto the first section and which extends towards the connection point at an angle to the longitudinal axis. This section preferably extends at an acute angle as been described beforehand. On account of the first sections, one succeeds in the second sections and the connection point being distanced to the valve leaflets and the commissures in the axial direction to such an extent that the movement of the valve leaflets is not compromised on opening and closing. The oblique or angled second sections, as has been described above, serve for reducing the valve frame in its diameter when it is pulled into the catheter. The structure holding bows can be configured such that they merely comprise the first section and instead of the second section then have the connecting connection members which create a connection to the commissure holding bows. The connection to the commissure holding bows is preferably effected in their first section or in the transition region between the first and the second section.

The commissure holding bows and/or the structure holding bows are further preferably each configured as single-part webs. Herein, the webs preferably have a greater cross section than the remaining webs of the lattice structure of the valve frame. Hence the holding bows can be configured in a stiffer manner in order to be able to generate a sufficient radially inwardly directed force for the folding together the lattice structure. The holding bows are herewith each formed by a web. Alternatively, the holding bows could however also be formed of several webs which for example extend in parallel. Since the holding bows lie in the free flow cross section through the aortic valve prosthesis, it is preferable for the holding bows to be configured as narrowly as possible transverse to the flow direction, so that they form a low as possible flow resistance to the blood.

The commissure holding bows are preferably each configured as one piece with the structure of the connected commissure, and/or the structure holding bows are preferably each configured as one-piece with the connected valve protection structure. Thus the holding bows can be formed as one piece together with the remaining structure of the valve frame, for example be cut from a pipe. By way of this, one can realise a particularly high firmness and at the same time a simple manufacture is rendered possible.

The connection point can be situated centrally with respect to the middle axis of the aortic valve prosthesis. An essentially symmetrical configuration is thus achieved, and such a configuration permits a uniform folding-together of the valve frame coming from the connection point. However, it is also conceivable to position the connection point in a manner radially distanced, which is to say eccentrically, to the middle axis of the aortic valve prosthesis. This can be advantageous in order to create an adequate free space in the central region, said free space permitting a later introduction of a further aortic valve prosthesis into the inside of the aortic valve prosthesis.

The connection point is preferably distanced in the axial direction from the plane in which the ends of the commissures are situated and preferably from the plane in which the free edges of the valve leaflets are situated. Hence the movability of the valve leaflets and the function of the aortic valve prosthesis are not compromised by the holding bows and the connection point.

Apart from the aforedescribed aortic valve prosthesis, the subject-manner of the invention is an assembly tool which is suitable for the implantation of aortic valve prosthesis as has been described above. The aortic valve prosthesis and the matching assembly tool permit a new type of implantation method which allows the repositioning of the aortic valve prosthesis on implantation by way of the aortic valve prosthesis being gripped again and folded together again, as has been described above. The assembly tool which is provided for this comprises a catheter which receives the aortic valve prosthesis and a handling element which is linearly movable in the inside of the catheter. The handling element is movable in a manner such that the aortic valve prosthesis can be pushed out of an open end of the catheter, but also, as has been described above, such that it can be pulled back into the opening again, wherein the holding bows slide on the edge of the opening in the previously described manner, by which means the aortic valve prosthesis is folded together which is to say radially pressed together again. The handling element at its free end comprises an engagement element which is configured in a manner such that it can come into releasable engagement with the holding element of the valve frame. The engagement element can thus be released from the holding element when the aortic valve prosthesis is correctly positioned. Particularly preferably, the holding element and the engagement element are configured in a manner corresponding to one another such that they can repeatedly come into releasable engagement. This means that the engagement element and the holding element are preferably configured such that the holding element can be gripped via the engagement element in order to thus be able to move the aortic valve prosthesis into the catheter again and possibly reposition it.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of an aortic valve prosthesis according to the invention;

FIG. 2 is a valve frame of an aortic valve prosthesis according to a second embodiment;

FIG. 3 is a sectioned view of the valve frame according to FIG. 2;

FIG. 4 is a valve frame of an aortic valve prosthesis according to a third embodiment; and

FIG. 5 is a sectioned view of the valve frame according to FIG. 4.

### DESCRIPTION OF PREFERRED EMBODIMENTS

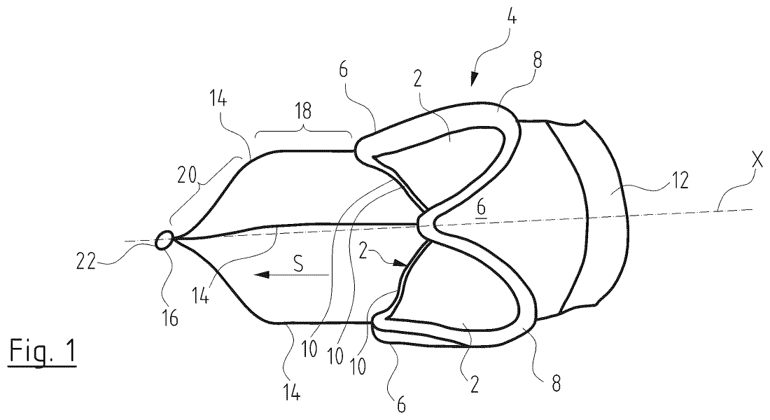

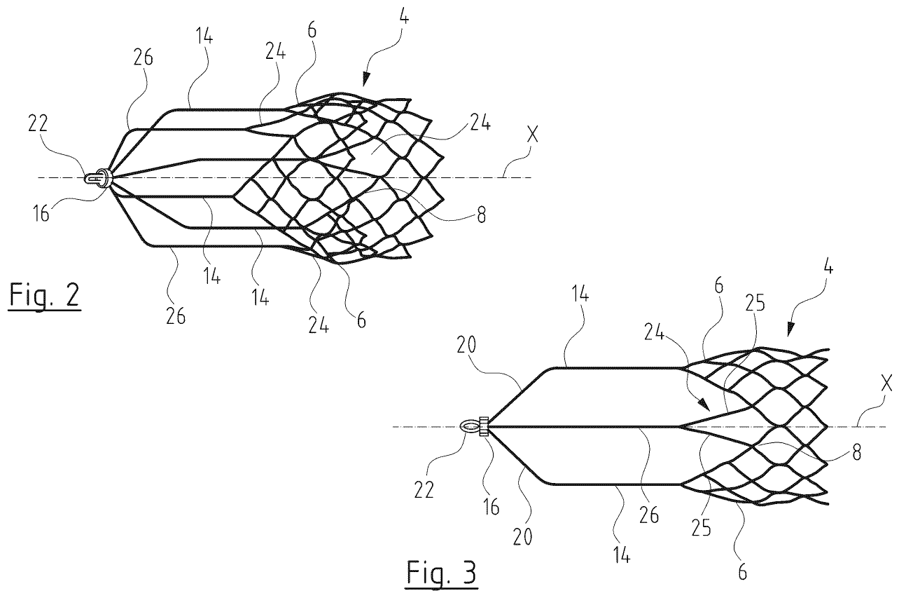

Referring to the drawings, FIG. 1 schematically shows the overall construction of an aortic valve prosthesis according to the invention. This aortic valve prosthesis is configured in order to replace the aortic valve in the heart in the course of a transcatheter implantation. The shown aortic valve prosthesis is configured as a semilunar valve with three valve leaflets 2. The valve leaflets 2 which can be manufactured for example of biological or synthetic material are fastened to a valve frame 4. The valve frame 4, as is explained in more detail by way of FIGS. 2 to 5, are configured as a lattice structure of metal which can be folded together in a manner such that the aortic valve prosthesis can be implanted through a catheter. The valve frame 4 defines three commissures 6 which are connected to one another by connection arches 8. The connection arches 8 herein connect the commissures 6 at their ends which are upstream seen in the flow direction S. The valve leaflets 2 are fastened, for example sewn, to the commissures 6 and the connection bows 8. The valve leaflets 2 at their free edges 10 sealingly bear on one another in the closed state. The edges 10 are distanced to one another in the opened state.

At its axial end which is away from the edges 10 in the direction of the longitudinal axis X, i.e. at the upstream end, the aortic valve prosthesis comprises a fastening region 12 with which it can be clampingly fastened in a blood vessel, in particular in the region of the natural valve ring. For this, the fastening region 12 is pressed radially outwards so that it comes to clampingly bear on the wall of the blood vessel. This widening can be achieved in the known manner by way of elastic restoring forces of the valve frame 4 or for example by way of a balloon which is placed in the inside. Inasmuch as this is concerned, the previously described aortic valve prosthesis corresponds to a known configuration. The fastening region 12 can also be configured in a manner in which it is force-decoupled with respect to the part of the valve frame 4 which forms the commissures 6, so that the fastening region 12 can deform without leading to undesirable deformations of the commissures 6 and the valve leaflets 2. According to the invention, the valve frame 4 comprises three commissure holding bows 14 which extend coming from the commissures 6. The commissure holding bows 14 are configured as single-part metallic bows which extend into the axial extension of the commissures 6 to a common connection point 16. This common connection point 16 is distanced to the axial ends of the commissures 6 and also to the edges 10 of the valve leaflets 2, in the axial direction X in the flow direction S, so that the function of the valve leaflets 2 is not compromised by the commissure holding bows 14. The commissure holding bows 14 each comprise a first section 18 and a second section 20. The first section 18 extends axially, which is to say essentially parallel to the longitudinal axis X in the extension of the adjacent commissure 6. The second section 20 coming from the first section 18 extends to the connection point 16 at an acute angle, which is to say an angle of less than 90° to the longitudinal axis X.

The commissure holding bows 14 are connected to a holding element 22, here in the form of an eyelet, at the connection point 16. The aortic valve prosthesis can be gripped at the holding element 22 and be pulled into a catheter in the direction of the longitudinal axis X. Herein, the second sections 20 come into bearing contact with the edge of a catheter opening, so that they are pressed radially inwards on account of their oblique course to the longitudinal axis X. Hence the commissure holding bows 14 are pressed radially inwards and in this manner the complete aortic valve prosthesis can be reduced in its diameter or cross section transverse to the longitudinal axis X. The fastening region 12 can therefore also be brought out of contact with the surrounding tissue so that a repositioning and removal of the aortic valve prosthesis becomes possible.

FIGS. 2 to 5 show further examples for valve frames 4. In these views, it is only the valve frame 4 which is without a textile sheathing and without the valve leaflets 2 which is shown.

The valve frame 4 which is shown in the FIGS. 2 and 3 corresponds essentially to the valve frame 4 which is described by way of FIG. 1, but additionally comprises a valve protection structure for the protection of the valve leaflets 2 on folding together. FIG. 3 shows a sectioned view of the valve frame 4 in FIG. 2, in which only two commissures 6 and an intermediate connection arch 8 is to be seen. In the representation of the valve frame 4, one can recognise that the valve frame 4 as a whole is configured as a metallic lattice structure, wherein the commissure holding bows 14 are configured as one piece with the remaining lattice structure and as a whole with the lattice structure which defines the commissures 6 and the connection arches 8. Such a lattice structure can be cut for example from a metallic pipe. Particularly preferably, shape-memory alloys such as for example nitinol are applied as a metal.

A valve protection structure 24 is arranged on each of the connection arches 8. The valve protection structures 24 each extend from the lowest point of the connection arches 8 in the axial direction, which is to say essentially parallel to the longitudinal axis X. In the shown example, each valve protection structure 24 is formed by two webs 25 which, coming from a connection arch 8, each taper to one another in a V-shaped manner and unify in a structure holding bow 26. The transition region between the webs 25 and the structure holding bow 26 lies roughly at the axial height of the tips of the commissures 6. Herein, the webs 25 and thus the tips of the valve protection structures 24 are distanced to the commissures 6 in the circumferential direction, so that the commissures 6 can moved independently of the valve protection structures 24. The structure holding bows 26 have a similar shape to the commissure holding bows 14 and are likewise connected to the connection point 16. The structure holding bows 26 with the valve protection structures 24 which connect thereon protect the valve leaflets 2 on folding together and have the effect that the middle regions of the valve leaflets 2 are moved radially inwards on folding together or pressing together the aortic valve prosthesis, without being clamped between the commissures 6 which move towards one another. The webs 25 of the valve protection structures 24 herein come to bear on the surfaces of the valve leaflets 2 in their middle region between the commissures 6. Particularly preferably, the structure holding bows 26 are shaped such that these move radially inwards in front of the commissure holding bows 14 on folding together the valve frame 4, so that the valve protection structures 24 move radially inwards before the commissures 6 move radially inwards. In such a manner, a defined folding of the valve leaflets 2 can be realised.

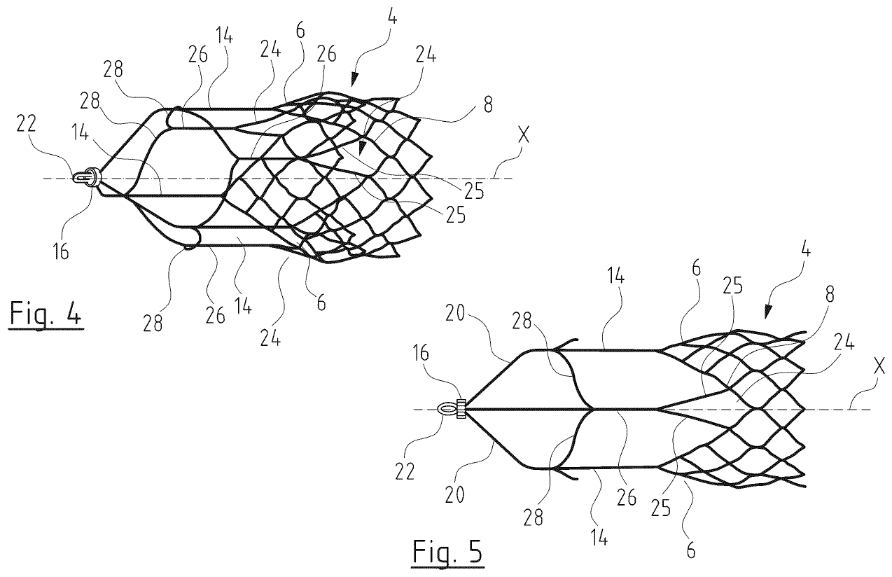

FIGS. 4 and 5 show a further embodiment example for a valve frame 4 of an aortic valve prosthesis according to the invention. The valve frame 4 which is shown in FIGS. 4 and 5 corresponds essentially to the valve frame which is shown in FIGS. 2 and 3 and it is only the structure holding bows 26 which are configured differently to the embodiment example according to FIGS. 2 and 3. Regarding all other features, the previous description is referred to. In the embodiment example according to FIGS. 4 and 5, the valve protection structures 24 are also formed by two webs 25 which extend to one another in a V-shaped manner, wherein the two webs 25 each merge into a structure holding bow 26 which extends parallel to the longitudinal axis X in the axial direction. In contrast to the preceding embodiment example, the three structure holding bows 26 however do not extend up to the connection point 16 of the commissure holding bows 14 but each divide into two connection members 28. This means that the structure holding bows 26 end in front of a connection point 16 in the axial direction. The connection members 28 connect onto the axial end of the structure holding bows 26 which is away from the webs 25. The connection members 28 extend essentially in the circumferential direction from a structure holding bow 26 to a commissure holding bow 14. In contrast to the second embodiment example according to FIGS. 2 and 3, this shaping permits a larger free cross section in the valve frame 4. Furthermore, the shaping has the effect that given a pressure upon the second sections 20 of the commissure holding bows 14 via the connection members 28, the valve protection structures 24 are moved radially inwards before the commissures 6 move radially inwards, so that the valve leaflets 2 fold in a defined manner.

The aortic valve prosthesis according to the invention thus comprises a valve frame 4 which permits a repositioning on implantation since the valve frame 4 can be re-gripped at the holding element 22 and can be pulled into a catheter. Herein, the complete valve frame 4 is pressed or folded together again in a radial manner via the commissure holding bows 14 and possibly the structure holding bows 26, so that it reduces in its diameter or cross section transverse to the longitudinal axis X and can be positioned anew the inside of a vessel. For this, a tool is preferably provided, said tool comprising an engagement element which is configured in a manner corresponding to the holding element 22, in order to come into releasable engagement with this.

While specific embodiments of the invention have been shown and described in detail to illustrate the application of the principles of the invention, it will be understood that the invention may be embodied otherwise without departing from such principles.

### LIST OF REFERENCE NUMERALS

2 valve leaflet
4 valve frame
6 commissures
8 connection arches, frame arches
10 edges of the valve leaflets 2
12 fastening region
14 commissure holding bows
16 connection point
18 first section
20 second section
22 holding element
24 flap protection structure
25 webs
26 structure holding bow
28 connection members
S flow direction
X longitudinal axis

1. An aortic valve prosthesis which is for a transcatheter implantation, the aortic valve prosthesis comprising:
three valve leaflets; and
a valve frame which can be folded together; and which valve frame comprises:
three commissures, between each of which one of the three valve leaflets is arranged
commissure holding bows, wherein the commissures at their ends each merge into one of the commissure holding bows, wherein the ends of the commissure holding bows which are away from the commissures are connected to one another in a common connection point; and
a holding element, arranged on the connection point to form a free end of the aortic valve prosthesis, said holding element being configured such that the holding element can be gripped by a tool.

2. An aortic valve prosthesis according to claim 1, wherein the holding element is provided with at least one undercut.

3. An aortic valve prosthesis according to claim 1, wherein the valve frame, in a region between two adjacent commissures comprises at least one valve protection structure which is configured such that the valve protection structure moves radially inwards on folding together the valve frame.

4. An aortic valve prosthesis according to claim 3, wherein the valve protection structure extends coming from a frame arch which connects the two adjacent commissures, and comprises an end which is distanced to the frame arch, said end being situated between tips of two commissures.

5. An aortic valve prosthesis according to claim 3, wherein the valve protection structures are each formed by one or more webs of a lattice structure of the valve frame.

6. An aortic valve prosthesis according to claim 3, wherein the valve protection structures each at one end are connected to a structure holding bow which extends to the connection point.

7. An aortic valve prosthesis according to claim 6, wherein the commissure holding bows and/or the structure holding bows each in at least one section extend to a longitudinal axis of the aortic valve prosthesis at an angle of smaller than 90 degrees.

8. An aortic valve prosthesis according to claim 6, wherein the commissure holding bows and/or the structure holding bows each comprise a first section which extends in the extension of the connected commissure or valve protection structure, and a connecting second section which extends to the connection point at an angle to the longitudinal axis.

9. An aortic valve prosthesis according to claim 6, wherein the commissure holding bows and/or the structure holding bows are each designed configured as one-part webs.

10. An aortic valve prosthesis according to claim 1, wherein the commissure holding bows are each configured as one piece with the structure of the connected commissure, and/or the structure holding bows are each formed as one piece with the connected valve protection structure.

11. An aortic valve prosthesis according to claim 1, wherein the connection point is situated centrally or radially distanced to a middle axis of the aortic valve prosthesis.

12. An aortic valve prosthesis according to claim 1, wherein the connection point is distanced in an axial direction from a plane in which the ends of the commissures are situated.

13. An assembly tool for the implantation of an aortic valve prosthesis according to claim 1, further comprising
a catheter which receives the aortic valve prosthesis; and
a handling element which is linearly movable inside of the catheter, wherein the handling element at a free end thereof comprises an engagement element which is configured such that the handling element can come into a releasable engagement with the holding element of the valve frame.

14. An aortic valve prosthesis according to claim 2, wherein the holding element is configured as a hook, ball or eyelet.

15. An aortic valve prosthesis according to claim 4, wherein the end of the valve protection structure is situated between the tips of two commissures without a direct connection to the commissures.

16. An aortic valve prosthesis according to claim 7, wherein the angle is smaller than 60 degrees.

17. An assembly tool according to claim 13, wherein the handling element can be repeatedly releasably engaged with the holding element.
